(19) United States
(12) Patent Application Publication
(10) Pub. No.: US 2021/0401269 A1
(43) Pub. Date: Dec. 30, 2021

(54) ANTENNA HOLDER AND CAPSULE ENDOSCOPE SYSTEM

(71) Applicant: OLYMPUS CORPORATION, Tokyo (JP)

(72) Inventors: Yuya TANAKA, Tokyo (JP); Ryosuke NAKAMURA, Tokyo (JP); Kaito OTONARI, Tokyo (JP)

(73) Assignee: OLYMPUS CORPORATION, Tokyo (JP)

(21) Appl. No.: 17/470,520

(22) Filed: Sep. 9, 2021

Related U.S. Application Data
(63) Continuation of application No. PCT/JP2019/013869, filed on Mar. 28, 2019.

(51) Int. Cl. A61B 1/00; H01Q 1/27; H04N 7/18; A61B 1/04

(52) U.S. Cl. CPC A61B 1/00016; H01Q 1/273; A61B 1/00045; A61B 1/041; H04N 7/183

(57) ABSTRACT

An antenna holder includes: a plurality of antenna holding portions configured to hold reception antennas configured to receive a radio signal transmitted from a capsule endoscope; a main body to which the antenna holding portions are fixed; a stretch portion that is mounted on the main body, the stretch portion being configured to cover all of the antenna holding portions; and a belt portion that is attached to the main body across the stretch portion in a first direction.

### CROSS-REFERENCE TO RELATED APPLICATION

This application is a continuation of International Application No. PCT/JP2019/013869, filed on Mar. 28, 2019, the entire contents of which are incorporated herein by reference.

### BACKGROUND

### 1. Technical Field

The present disclosure relates to an antenna holder that holds a reception antenna that receives a radio signal, and a capsule endoscope system.

### 2. Related Art

In the related art, there is a known capsule endoscope having an imaging function, a wireless communication function, or the like that is embedded inside a capsule shaped casing that is formed with a size so as to be capable of being introduced into the digestive tract of a subject, such as a patient. After the capsule endoscope is swallowed from the mouth of the subject, the capsule endoscope generates image data by sequentially capturing images inside the subject while moving inside the subject, such as inside the digestive tract, by a peristaltic movement, and sequentially and wirelessly transmits the image data. The image data that is wirelessly transmitted by the capsule endoscope in this way is received by a reception device via a plurality of reception antennas that are provided outside the subject. Each of the reception antennas is held by a sheet shaped holding portion and fixed to the surface of the subject (for example, see Japanese Patent 5193402).

With the antenna holder described in Patent Document 1, the holding portion is attached to the subject by using a stretch portion and a belt that have a stretchable property. The stretch portion and the belt are connected to the holding portion at one end and another end thereof in the circumference direction of the subject. In Japanese Patent 5193402, the holding portion is brought into close contact with the subject by expansion and contraction of the stretch portion. Consequently, the reception antennas are brought into close contact with the subject via the holding portion.

### SUMMARY

In some embodiments, an antenna holder includes: a plurality of antenna holding portions configured to hold reception antennas configured to receive a radio signal transmitted from a capsule endoscope; a main body to which the antenna holding portions are fixed; a stretch portion that is mounted on the main body, the stretch portion being configured to cover all of the antenna holding portions; and a belt portion that is attached to the main body across the stretch portion in a first direction.

In some embodiments, a capsule endoscope system includes: a capsule endoscope configured to be introduced into a subject; an antenna holder configured to be attached to the subject, the antenna holder being configured to hold a plurality of reception antennas; an antenna device that is mounted on the antenna holder and that includes the plurality of reception antennas configured to receive a radio signal transmitted from the capsule endoscope; a reception device that is detachably connected to the antenna device, the reception device being configured to record or display data of the radio signal by performing a predetermined process on the radio signal received by the antenna device; and an image processing apparatus configured to perform a process on or display image data of an image inside the subject captured by the capsule endoscope. The antenna holder includes a plurality of antenna holding portions configured to hold the reception antennas, a main body to which the antenna holding portions are fixed, a stretch portion that is mounted on the main body, the stretch portion being configured to cover all of the antenna holding portions, a belt portion that is attached to the main body across the stretch portion in a first direction.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of a capsule endoscope system that includes an antenna holder according to a first embodiment of the disclosure;

FIG. 2 is a schematic diagram illustrating a schematic configuration of the antenna holder illustrated in FIG. 1;

FIG. 3 is a schematic diagram illustrating a schematic configuration of the reception antenna illustrated in FIG. 1;

FIG. 4 is a magnified view of the antenna holder illustrated in FIG. 2;

FIG. 5 is a diagram for explaining a load applied to the subject when the antenna holder is attached;

FIG. 6 is a diagram illustrating an example (a first example) in a case in which the antenna holder is attached to the subject;

FIG. 7 is a diagram illustrating an example (a second example) in a case in which the antenna holder is attached to the subject;

FIG. 8 is a diagram illustrating a configuration of an antenna holder according to a second embodiment of the disclosure;

FIG. 9 is a diagram illustrating a configuration of an antenna holder according to a third embodiment of the disclosure;

FIG. 10 is a diagram illustrating a configuration of an antenna holder according to a fourth embodiment of the disclosure;

FIG. 11 is a diagram illustrating a configuration of an antenna holder according to a fifth embodiment of the disclosure;

FIG. 12 is a diagram illustrating a configuration of an antenna holder according to a sixth embodiment of the disclosure;

FIG. 13 is a diagram illustrating a configuration of an antenna holder according to a seventh embodiment of the disclosure;

FIG. 14A is a diagram illustrating a configuration of an antenna holder according to an eighth embodiment of the disclosure;

FIG. 14B is a diagram illustrating a configuration of an antenna holder according to the eighth embodiment of the disclosure;

FIG. 15 is a diagram illustrating a configuration of an antenna holder according to the eighth embodiment of the disclosure;

FIG. 16A is a diagram illustrating a configuration of an antenna holder according to a ninth embodiment of the disclosure;

FIG. 16B is a diagram illustrating a configuration of an antenna holder according to the ninth embodiment of the disclosure;

FIG. 17A is a diagram illustrating a configuration of an antenna holder according to a modification of the ninth embodiment of the disclosure; and

FIG. 17B is a diagram illustrating a configuration of an antenna holder according to a modification the ninth embodiment of the disclosure.

### DETAILED DESCRIPTION

In the following, a capsule endoscope system that uses a capsule endoscope will be described as preferred embodiments according to the disclosure. Furthermore, in the drawings used for the following description, components that are identical to those in embodiments are assigned the same reference numerals. Furthermore, the drawings used for the descriptions below are only schematic illustrations, and the relationship between the thickness and the width of each member, the proportions of each member, and so on are different from those used in practice.

### First Embodiment

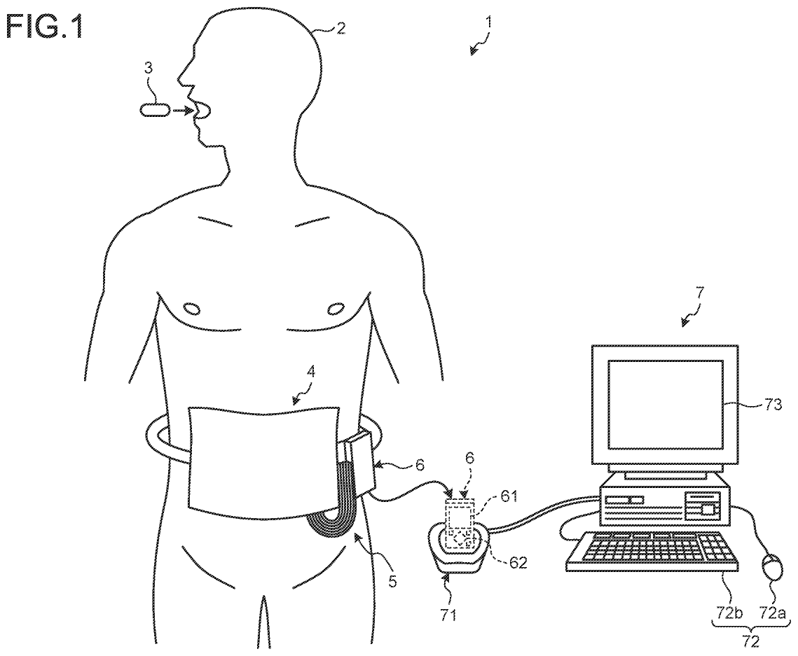

FIG. 1 is a schematic diagram illustrating a schematic configuration of a capsule endoscope system that includes an antenna holder according to a first embodiment of the disclosure. A capsule endoscope system 1 illustrated in FIG. 1 includes a capsule endoscope 3 as a medical device configured to be introduced into a subject 2; an antenna holder 4 that is attached to the subject 2 and that holds a plurality of reception antennas; an antenna device 5 that is attached to the antenna holder 4 and that has the plurality of reception antennas configured to receive a radio signal transmitted from the capsule endoscope 3 that is introduced into the subject 2; a reception device 6 that is connected to the antenna device 5 so as to be freely attached to and removed from the antenna device 5 and that records or displays data by performing a predetermined process on the radio signal received by the antenna device 5; and an image processing apparatus 7 that performs a process on and/or displays associated with the image data related to inside the subject 2 captured by the capsule endoscope 3. The antenna holder 4 and the reception antennas included in the antenna device 5 constitute an antenna unit.

The capsule endoscope 3 has an imaging function for capturing an image inside the subject 2 and a wireless transmission function for transmitting a radio signal including the image data obtained by capturing images inside the subject 2 to reception antennas 51. The capsule endoscope 3 passes through an esophagus inside the subject 2 by being swallowed into the subject 2 and moves inside the subject 2 by a peristaltic movement of a lumen of a digestive tract. The capsule endoscope 3 sequentially captures images inside the subject 2 at minute time intervals of, for example, 0.5 seconds (2 fps) while moving inside the subject 2, generates image data of the images captured inside the subject 2, and sequentially transmits the generated pieces of image data to the antenna device 5. The capsule endoscope 3 outputs a radio signal at frequencies between, for example, 300 MHz and 500 MHz.

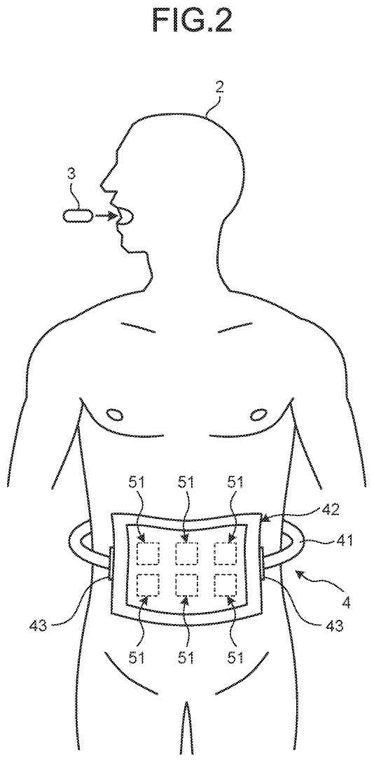

FIG. 2 is a schematic diagram illustrating a schematic configuration of the antenna holder illustrated in FIG. 1 and is a diagram illustrating a state in which the antenna holder 4 is attached to the subject 2. The antenna holder 4 includes, as illustrated in FIG. 2, a belt portion 41 that fixes the antenna holder 4 to the subject 2, an antenna mounting portion 42 on which a plurality of reception antennas 51 are mounted, and connecting portions 43 that connect the belt portion 41 and the antenna mounting portion 42.

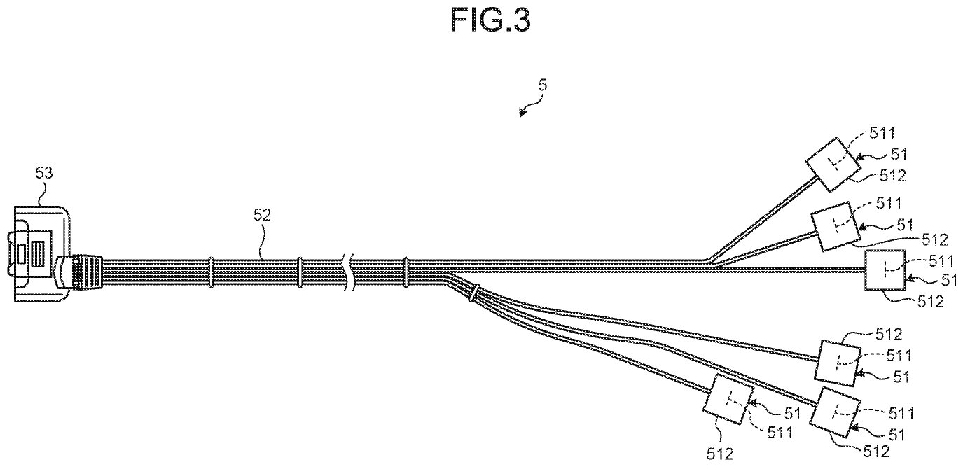

In the following, a configuration of the antenna device 5 will be described with reference to FIG. 3. FIG. 3 is a schematic diagram illustrating a schematic configuration of the reception device illustrated in FIG. 1. As illustrated in FIG. 3, the antenna device 5 includes the plurality of the reception antennas 51 each of which receives a radio signal from the capsule endoscope 3, an antenna cable 52 that propagates the radio signal received by the plurality of the reception antennas 51 to the reception device 6, and a connector portion 53 that is connected to the reception device 6. A configuration of the reception antennas 51 will be described later.

A description will be given here by referring back to FIG. 2. The belt portion 41 included in the antenna holder 4 fixes the antenna holder 4 to the subject 2 along the lines of the surface of the body of the subject 2 by being attached to the subject 2. It is preferable that the belt portion 41 be formed by using a stretchable material, such as a rubber or polyurethane elastic fiber, that is able to fix the antenna holder 4 to the subject 2 in accordance with a physical size of the subject.

The connecting portion 43 is formed by using a rigid member. The connecting portion 43 has a ring shape, is connected to the belt portion 41 on one end side, and is connected to the antenna mounting portion 42 (a main body 421 that will be described later) on the other end side.

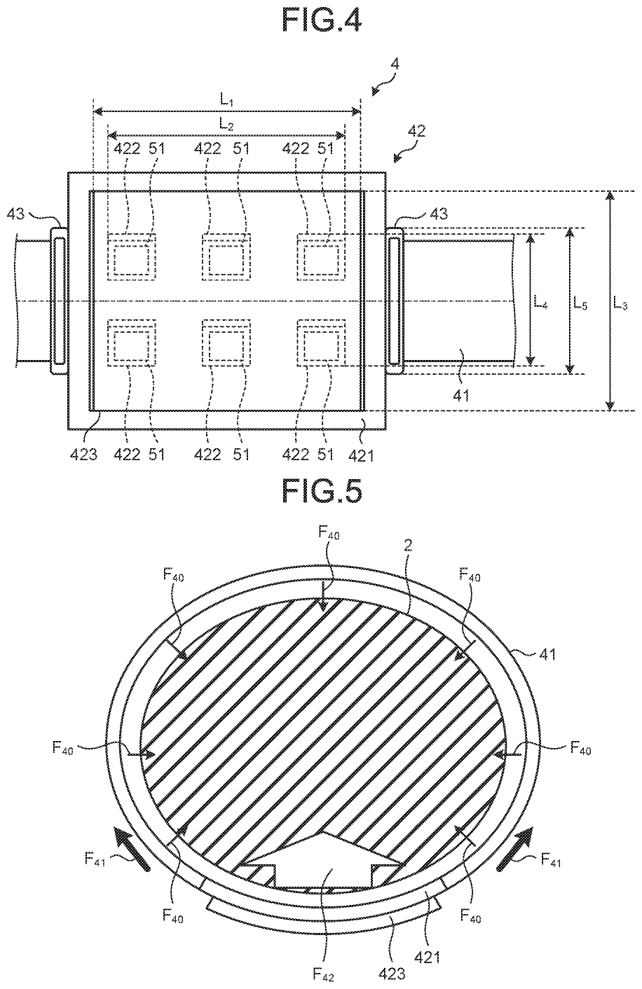

FIG. 4 is a magnified view of the antenna holder 4 illustrated in FIG. 2. The antenna mounting portion 42 includes the main body 421 that accommodates the plurality of the reception antennas 51; six antenna holding portions 422 that are disposed on the main body 421, that hold the respective reception antennas 51, and that fix the location of the respective reception antennas 51; and a stretch portion 423 that is disposed on the main body 421 and that has a stretchable property. The following description is based on the assumption that the number of the antenna holding portions 422 are six and the six reception antennas 51 are mounted; however, the number of the antenna holding portions 422 and the reception antennas 51 is not limited to six as long as one or more of the antenna holding portions 422 and the reception antennas 51 may be used. Furthermore, the number of the antenna holding portions 422 and the number of the reception antennas 51 are not always be the same.

The main body 421 is formed by using cloth or the like. If the main body 421 has, for example, a bag shape, the main body 421 has an openable and closeable opening that is formed by using a snap fastener, a zip fastener, or a hook-and-loop fastener.

Each of the antenna holding portions 422 is formed by using cloth or the like having a pocket shape and contains therein one of the reception antennas 51. Each of the antenna holding portions 422 is disposed in association with the position in which the associated reception antenna 51 is arranged. Furthermore, in the first embodiment, an example in which the antenna holding portions 422 are disposed on a first surface of the main body 421, the first surface of the main body 421 being a surface opposite to a second surface of the main body 421, the second surface of the main body 421 being a surface on a side of the subject 2, will be described; however, the antenna holding portions 422 may also have a configuration in which the antenna holding portions 422 are disposed on the second surface of the main body 421.

The stretch portion 423 is formed by using a material having a stretchable property, such as a rubber or polyurethane elastic fiber. If the antenna holder 4 is attached to the subject 2, the stretch portion 423 is arranged on the side opposite to the subject 2 side relative to the main body 421. Namely, the stretch portion 423 covers the surface on the subject 2 side relative to the reception antenna 51. The stretch portion 423 has a uniform elastic force. The stretch portion 423 stretches in accordance with a tensile force that is applied to the main body 421 and that is generated by the belt portion 41 being attached to the subject.

In the first embodiment, the connecting portions 43 are disposed at both ends of the main body 421 in a first direction (a horizontal direction illustrated in FIG. 4). Namely, the belt portion 41 is connected to the main body 421 across the stretch portion 423 in the first direction.

The stretch portion 423 is formed having a size that covers an area including all of the antenna holding portions 422. For example, in the example illustrated in FIG. 4, a length L₁ of the first direction (the horizontal direction in FIG. 4) of the stretch portion 423 is greater than a maximum length L₂ between the antenna holding portions 422 in the first direction. Here, the first direction (the horizontal direction in FIG. 4) is a direction that connects the end portions at each of which the belt portion 41 is attached to the main body 421 and that corresponds to a direction in which a tensile force from the belt portion 41 is applied. It is preferable that a natural length of the stretch portion 423 in the first direction be smaller than the length L₁ of a mounting area of the stretch portion 423 mounted on the main body 421 in the first direction. In this case, the stretch portion 423 mounted on the main body 421 is in a state in which a compression force is applied to the stretch portion 423. Furthermore, the natural length mentioned here corresponds to a length of the stretch portion 423 that is in a natural state. The natural state mentioned here indicates a state in which a load other than gravity is not applied and a tensile force is not applied to the stretch portion 423.

Furthermore, in the example illustrated in FIG. 4, a length L₃ of the stretch portion 423 in a second direction (a vertical direction in FIG. 4) is greater than a maximum length L₄ between the antenna holding portions 422 in the second direction. Here, the second direction is a direction that is orthogonal to the first direction. Furthermore, in the first embodiment, a length L₅ of each of the connecting portions 43 in the second direction is smaller than the length L₃ and is greater than the maximum length L₄.

Furthermore, both ends of the stretch portion 423 in the first direction is fixed to the main body 421.

The reception antennas 51 are constituted by substrates 512 each having mounted thereon antenna elements 511 each of which receives a radio signal output from the capsule endoscope 3. The substrates 512 may also be constituted by using bendable and flexible substrates or may also be constituted by using rigid substrates having rigidity. The antenna elements 511 are constituted by using antennas, such as dipole antennas.

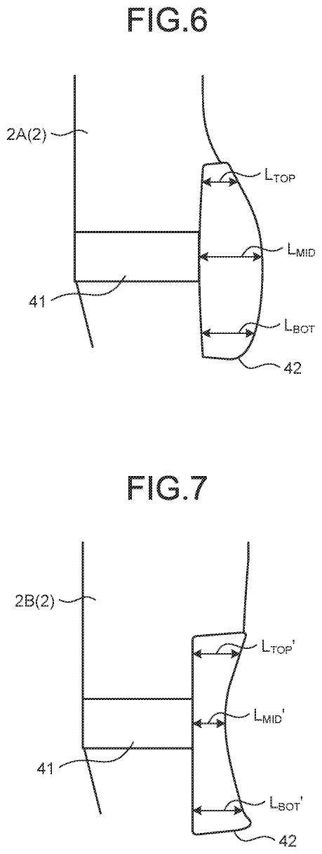

In the following, a load applied to the subject 2 when the antenna holder 4 is attached to the subject 2 and a function of the stretch portion 423 will be described with reference to FIGS. 5 to 7. FIG. 5 is a diagram for explaining a load applied to the subject when the antenna holder is attached. FIG. 6 is a diagram illustrating an example (a first example) in a case in which the antenna holder is attached to the subject. FIG. 7 is a diagram illustrating an example (a second example) in a case in which the antenna holder is attached to the subject.

If the antenna holder 4 is attached to the subject 2, a load F₄₀ corresponding to constriction is applied from the belt portion 41 to the subject 2 (see FIG. 5). At this time, a tensile force F₄₁ is applied to the main body 421 from the belt portion 41 via the connecting portions 43. If the tensile force F₄₁ is applied to the main body 421, the stretch portion 423 is elongated and the elongated stretch portion 423 applies a load F₄₂ that presses the main body 421 with respect to the subject 2. The main body 421 is brought into close contact with the subject 2 caused by the load F₄₂.

Furthermore, the stretch portion 423 is deformed in accordance with the shape of a lower abdominal region of the subject 2. FIGS. 6 and 7 are lateral views of the subject 2 illustrated in FIG. 2 viewed from the side.

For example, if the antenna holder 4 is mounted on a subject 2A having a convex shaped abdominal region (see FIG. 6), in a circumference direction of the abdominal region of the subject 2, a side-surface distance L_{NED} of the antenna mounting portion 42 located at the central part of the abdominal region is greater than a side-surface distance L_{TOP} of the antenna mounting portion 42 located on the chest part side of the abdominal region and a side-surface distance L_{BOT} of the antenna mounting portion 42 located on the leg part side of the abdominal region.

In contrast, if the antenna holder 4 is mounted on the subject 2A having a concave shaped abdominal region (see FIG. 7), in the circumference direction of the abdominal region of the subject 2, a side-surface distance L_{MID′} of the antenna mounting portion 42 located at the central part of the abdominal region is smaller than a side-surface distance L_{TOP′} of the antenna mounting portion 42 located on the chest part side of the abdominal region and a side-surface distance L_{BOT′} of the antenna mounting portion 42 located on the leg part side of the abdominal region.

In the first embodiment, even if the magnitude relationship of the side-surface distance of the subject 2 on which the antenna holder 4 is mounted varies, it is possible to bring the main body 421 into close contact with the subject 2 caused by elongation of the stretch portion 423.

A description will be given here by referring back to FIG. 1. The reception device 6 records image data that is related to inside the subject 2 and that is included in the radio signal transmitted from the capsule endoscope 3 via the plurality of the reception antennas 51 or displays an image that is associated with the image data that is related to inside the subject 2. The reception device 6 includes a reception display unit 61 that displays the image associated with the image data and an operating unit 62 that receives an indication signal for operating the reception device 6 and an input of information related to a location of each of the reception antennas 51.

Furthermore, the reception device 6 receives the radio signal transmitted from the capsule endoscope 3 via each of the reception antennas 51, calculates and records a reception intensity (received electric field intensity) of the received radio signal for each of the reception antennas 51, and estimates a location of the capsule endoscope 3 inside the subject 2. The reception device 6 records, in an associated manner, the image data included in the radio signal received from the capsule endoscope 3, the reception intensity of the radio signal received by each of the reception antennas 51, and time information on the image data generated by the capsule endoscope 3.

The image processing apparatus 7 displays an image associated with the image data on inside the subject 2 acquired via the reception device 6. The image processing apparatus 7 includes a cradle 71 that reads image data or the like from the reception device 6; an operation input unit 72, such as a mouse 72a and a keyboard 72b; and a display unit 73 that displays the image associated with the image data. The cradle 71 acquires, from the reception device 6 at the time of attachment of the reception device 6, image data, a reception intensity of each of the reception antennas 51 associated with the image data, the time information on the image data generated by the capsule endoscope 3, identification information on the capsule endoscope 3, and the like, and then, transfers the various kinds of acquired information to the image processing apparatus 7. The operation input unit 72 receives an input performed by a user. The user observes a living body region inside the subject 2, such as the esophagus, the stomach, the small intestine, the large intestine, while viewing, by operating the operation input unit 72, images of inside the subject 2 that are sequentially displayed by the image processing apparatus 7 and makes a diagnosis of the subject 2.

In the first embodiment described above, with the antenna holder 4 that holds the reception antennas 51, the stretch portion 423 disposed on the main body 421 is elongated in accordance with a body shape of the subject 2, so that the main body 421 is brought into close contact with the subject 2. According to the first embodiment, by allowing the main body 421 to be brought into close contact with the subject 2, is it possible to attach the reception antennas to the subject regardless of the body shape of the subject without reducing contact properties with respect to the subject.

### Second Embodiment

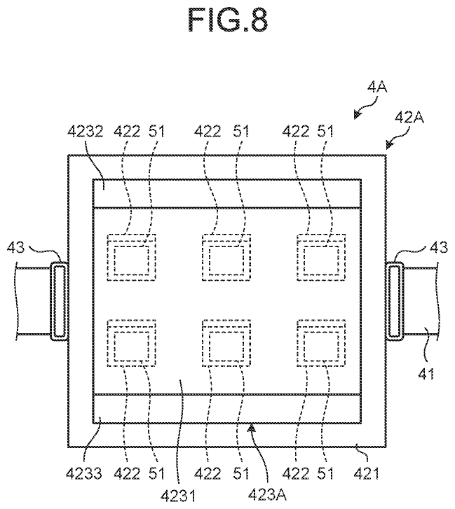

FIG. 8 is a diagram illustrating a configuration of an antenna holder according to a second embodiment of the disclosure. In the first embodiment described above, a case has been described as one example in which the elastic forces of the stretch portion 423 are uniform; however, in the second embodiment, elastic forces are partially different in a stretch portion 423A. An antenna holder 4A according to the second embodiment includes the belt portion 41, an antenna mounting portion 42A, and the connecting portions 43 that connects the belt portion 41 and the antenna mounting portion 42A. In the following, a configuration of a component (the antenna mounting portion 42A) that is different from that described above in the first embodiment will be described.

The antenna mounting portion 42A includes the above described main body 421, the above described antenna holding portions 422, and a stretch portion 423A that is disposed on the main body 421 and that has a stretchable property. In the following, the stretch portion 423A that is different from the stretch portion described above in the first embodiment will be described.

The stretch portion 423A is formed by using a material having a stretchable property, such as a rubber or polyurethane elastic fiber. Similarly to the stretch portion 423, if the antenna holder 4 is attached to the subject 2, the stretch portion 423A is arranged on the side opposite to the subject 2 side relative to the main body 421. The stretch portion 423A includes a first stretch portion 4231 that has a first elastic force, a second stretch portion 4232 and a third stretch portion 4233 that have a second elastic force that is greater than the first elastic force. The elastic force of the first elastic force is the same as the elastic force of, for example, the stretch portion 423 described above.

In the stretch portion 423A, the first stretch portion 4231 is formed having a size that covers an area including all of the antenna holding portions 422. The second stretch portion 4232 is disposed on one end side (the upper side in FIG. 8) of the first stretch portion 4231 in the second direction. The third stretch portion 4233 is disposed on the other end side (the lower side in FIG. 8) of the first stretch portion 4231 in the second direction.

Because the second stretch portion 4232 and the third stretch portion 4233 are disposed, in the second direction, the elastic force of the stretch portion 423A at the positions corresponding to the chest part side (the side-surface distance L_{TOP}) of the abdominal region and the leg part side (the side-surface distance L_{BOT}) of the abdominal region illustrated in FIG. 6 is higher than the elastic force of the first stretch portion 4231. In particular, the stretch portion 423A is able to increase constriction on the chest part side of the abdominal region and the leg part side of the abdominal region at the time of attaching to the subject 2A (see FIG. 6) having the convex shaped abdominal region.

In the second embodiment described above, in the antenna holder 4A that holds the reception antennas 51, the stretch portion 423A disposed on the main body 421 is elongated in accordance with the body shape of the subject 2, and allows the main body 421, in particular, an end portion on the chest part side and the leg part side, to be strongly brought into close contact with the subject 2. According to the second embodiment, it is possible to attach the reception antennas to the subject without decreasing contact properties with respect to the subject.

### Third Embodiment

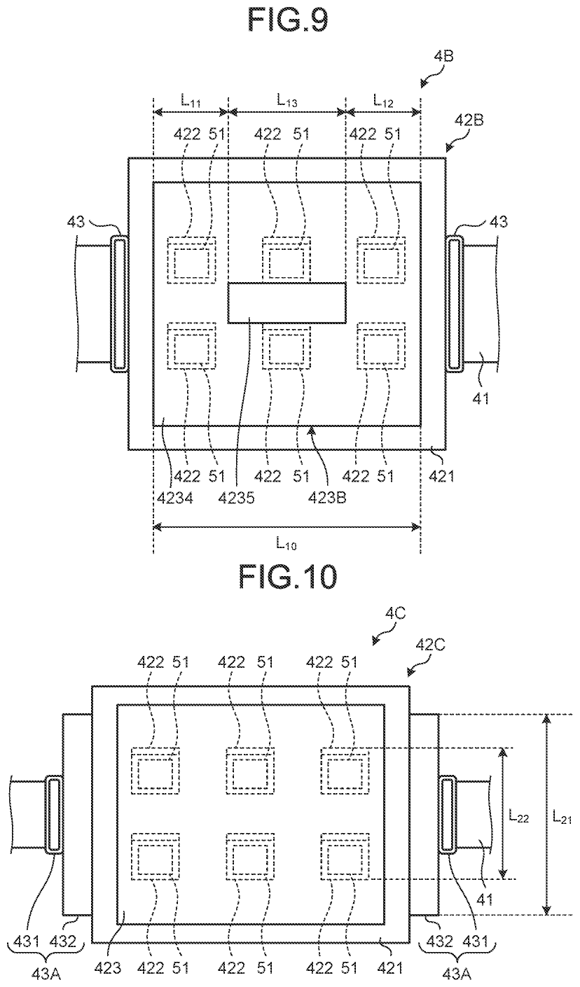

FIG. 9 is a diagram illustrating a configuration of an antenna holder according to a third embodiment of the disclosure. In the first embodiment described above, a case has been described as one example in which the elastic forces of the stretch portion 423 are uniform; however, in the third embodiment, elastic forces are partially different in a stretch portion 423B. An antenna holder 4B according to the third embodiment includes the belt portion 41, an antenna mounting portion 42B, and the connecting portions 43 that connect the belt portion 41 and the antenna mounting portion 42B. In the following, a configuration of a component (the antenna mounting portion 42B) that is different from that described above in the first embodiment will be described.

The antenna mounting portion 42B includes the above described main body 421, the above described antenna holding portions 422, and the stretch portion 423B that is disposed on the main body 421 and that has a stretchable property. In the following, the stretch portion 423B that is different from the stretch portion described above in the first embodiment will be described.

The stretch portion 423B is formed by using a material having a stretchable property, such as a rubber or polyurethane elastic fiber, and using a material having a non-stretchable property that does not have the stretchable property. Similarly to the stretch portion 423, if the antenna holder 4 is attached to the subject 2, the stretch portion 423B is arranged on the side opposite to the subject 2 side relative to the main body 421. The stretch portion 423B includes a base portion 4234 that has a stretchable property and a non-stretch portion 4235 that does not have a stretchable property. The elastic force of the base portion 4234 is the same as the elastic force of, for example, the stretch portion 423 described above. The non-stretch portion 4235 is disposed at the central part of the base portion 4234.

The base portion 4234 is formed having a size that covers an area including all of the antenna holding portions 422. The non-stretch portion 4235 is disposed at the central part of the base portion 4234 in the first direction and the second direction. Here, in the first direction (the horizontal direction in FIG. 9), if the maximum length of the base portion 4234 is denoted by L₁₀, a length between one end of the base portion 4234 and the non-stretch portion 4235 is denoted by L₁₁, a length between the other end of the base portion 4234 and the non-stretch portion 4235 is denoted by L₁₂, and a length of the non-stretch portion 4235 is denoted by L₁₃, the maximum length L₁₀ and a length L₁₁+L₁₂+L₁₃ in a natural state has a relationship denoted by L₁₀<L₁₁+L₁₂+L₁₃. If the above described relationship between the base portion 4234 and the non-stretch portion 4235 is satisfied, the elastic force at the position corresponding to the central part (the side-surface distance L_{MID′}) of the abdominal region is higher than the elastic force at the position corresponding to the chest part side (the side-surface distance L_{TOP′}) of the abdominal region and the leg part side (the side-surface distance L_{BOT′}) of the abdominal region. In particular, the stretch portion 423B is able to increase constriction at the central part of the abdominal region at the time of attaching to the subject 2B (see FIG. 7) having the concave shaped abdominal region.

In the third embodiment described above, in the antenna holder 4B that holds the reception antennas 51, the stretch portion 423B disposed on the main body 421 is elongated in accordance with the body shape of the subject 2, and allows the main body 421, in particular, the central part of the abdominal region, to be strongly brought into close contact with the subject 2. According to the third embodiment, it is possible to attach the reception antennas to the subject without decreasing contact properties with respect to the subject.

### Fourth Embodiment

FIG. 10 is a diagram illustrating a configuration of an antenna holder according to a fourth embodiment of the disclosure. In the first embodiment described above, a case has been described as one example in which the belt portion 41 and the antenna mounting portion 42 are connected via the connecting portions 43; however, in the fourth embodiment, a tensile force applied to the belt portion 41 is efficiently and uniformly transferred by using the antenna mounting portion 42C. An antenna holder 4C according to the fourth embodiment includes the belt portion 41, the antenna mounting portion 42C, and a connecting portions 43A that connects the belt portion 41 and the antenna mounting portion 42C. In the following, a configuration of components (the connecting portions 43A) that are different from that described above in the first embodiment will be described.

The connecting portions 43A includes first connecting portions 431 that are connected to the belt portion 41 and second connecting portions 432 one end of which is connected to the first connecting portions 431 and the other end of which is connected to the main body 421. The second connecting portions 432 correspond to a reinforcement portion.

The first connecting portions 431 are formed by using, similarly to the connecting portions 43 described above, a rigid member. Each of The first connecting portions 431 has a ring shape, is connected to the belt portion 41 at one end, and is connected to the second connecting portions 432 at the other end.

The second connecting portions 432 are formed by using a non-stretchable material, such as leather, resin, metal. The second connecting portions 432 have one of a columnar shape, a plate shape, and a sheet shape. Each of the second connecting portions 432 has a strip shape and a length L₂₁ thereof in the second direction is set to be greater than or equal to a minimum length L₂₂ of an arrangement area, in which all of the antenna holding portions 422 are arranged in the main body 421, in the second direction.

In the fourth embodiment described above, in the antenna holder 4C that holds the reception antennas 51, the stretch portion 423 disposed on the main body 421 is elongated in accordance with the body shape of the subject 2 and allows the main body 421 to be brought into close contact with the subject 2. At this time, the second connecting portions 432 having the non-stretchable property applies a tensile force of the belt portion 41 to a wide area in the second direction, so that a uniform load (tensile force) is transferred to the main body 421 and the stretch portion 423. According to the fourth embodiment, it is possible to obtain the same effect described in the first embodiment and transfer the tensile force applied from the belt portion 41 is more uniformly transferred to the antenna mounting portion 42C and it is thus possible to further ensure contact properties with respect to the subject 2.

### Fifth Embodiment

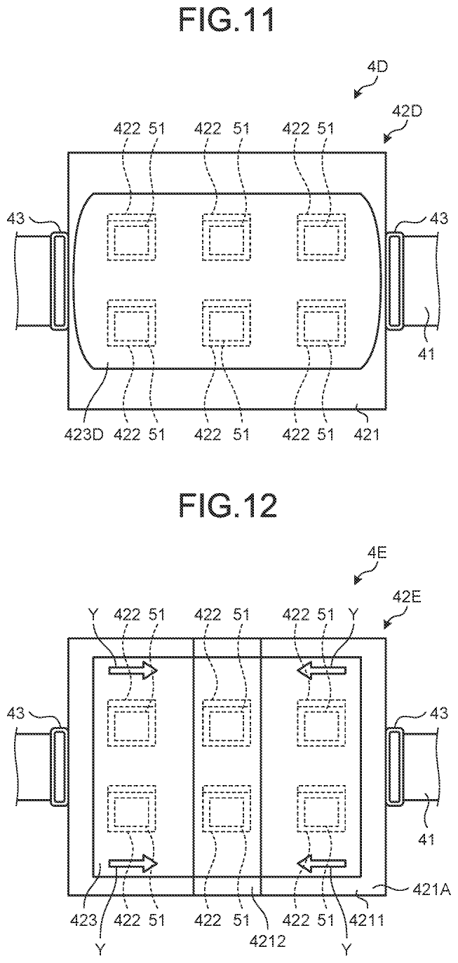

FIG. 11 is a diagram illustrating a configuration of an antenna holder according to a fifth embodiment of the disclosure. In the first embodiment described above, a case has been described as one example (for example, see FIG. 4) in which the outer shape of the stretch portion 423 is rectangular; however, in the fifth embodiment, a part of an outer rim of a stretch portion 423D has an arc shape. An antenna holder 4D according to the fifth embodiment includes the belt portion 41, an antenna mounting portion 42D, and the connecting portions 43. In the following, a configuration of a component (the antenna mounting portion 42D) that is different from that described above in the first embodiment will be described.

The antenna mounting portion 42D includes the above described main body 421, above described the antenna holding portions 422, and a stretch portion 423D that has a stretchable property and that is disposed on the main body 421. In the following, the stretch portion 423D that is different from the stretch portion described above in the first embodiment will be described.

The stretch portion 423D is formed by using a material having a stretchable property, such as a rubber or polyurethane elastic fiber. The stretch portion 423D has an arc shape on an outer rim (an outer rim closer to each of the connecting portions 43) that intersects the second direction. The stretch portion 423D has an arc shape curved on the side that is away from each of the connecting portions 43 as stretch portion 423D is away from the central part in the second direction. Consequently, the stretch portion 423D has a shape in which the length in the first direction is gradually reduced from the central part toward the end portion (upper and lower ends) in the second direction.

In the fifth embodiment described above, in the antenna holder 4B that holds the reception antennas 51, the stretch portion 423D is elongated in accordance with the body shape of the subject 2 and allows the main body 421 to be brought into close contact with the subject 2. At this time, the length between the upper end and the lower end of the stretch portion 423D in the first direction is shorter than the length of the central part; therefore, a strong tensile force is generated at the upper end and the lower end. Consequently, in particular, the end portions on the chest part side and the leg part side are strongly brought into close contact with the subject 2. According to the fifth embodiment, it is possible to attach the reception antennas to the subject without reducing contact properties with respect to the subject.

### Sixth Embodiment

FIG. 12 is a diagram illustrating a configuration of an antenna holder according to a sixth embodiment of the disclosure. In the sixth embodiment, a reinforcement portion 4212 that reinforces a main body 421A of an antenna mounting portion 42E is disposed. An antenna holder 4E according to the sixth embodiment includes the belt portion 41, the antenna mounting portion 42E, and the connecting portions 43. In the following, a configuration of a component (the antenna mounting portion 42E) that is different from that described above in the first embodiment will be described.

The antenna mounting portion 42E includes the above described antenna holding portions 422, and a main body 421A. In the following, the main body 421A that is different from the main body described above in the first embodiment will be described.

The main body 421A includes a base portion 4211 that has a bag shape formed by using cloth or the like and a reinforcement portion 4212 that is disposed at the base portion 4211. The base portion 4211 has an openable and closeable opening that is formed by using, for example, a snap fastener, a zip fastener, or a hook-and-loop fastener. In the sixth embodiment, the connecting portions 43 are disposed at both ends of the main body 421A in the first direction (the horizontal direction in FIG. 12). Namely, the belt portion 41 is connected to the main body 421A across the stretch portion 423 in the first direction.

The reinforcement portion 4212 is formed by using a non-stretchable material, such as leather. The reinforcement portion 4212 has a strip shape, is disposed at the center in the first direction, and extends in the second direction.

In the sixth embodiment described above, in the antenna holder 4E that holds the reception antennas 51, the reinforcement portion 4212 is also disposed on the main body 421A. Here, if the main body 421A is mounted on the subject 2, the elastic force of the stretch portion 423 is applied toward the central part (see an arrow Y illustrated in FIG. 12). Lines caused by the elastic force are likely to be generated at the central part of the main body 421A. According to the sixth embodiment, it is possible to obtain the same effect described in the first embodiment and it is also prevent, by arranging the reinforcement portion 4212, the lines from being generated on the main body 421A.

In the sixth embodiment, the reinforcement portion 4212 may also be disposed at a plurality of positions in accordance with the number of the reception antennas 51 or the disposition of the reception antennas 51.

### Seventh Embodiment

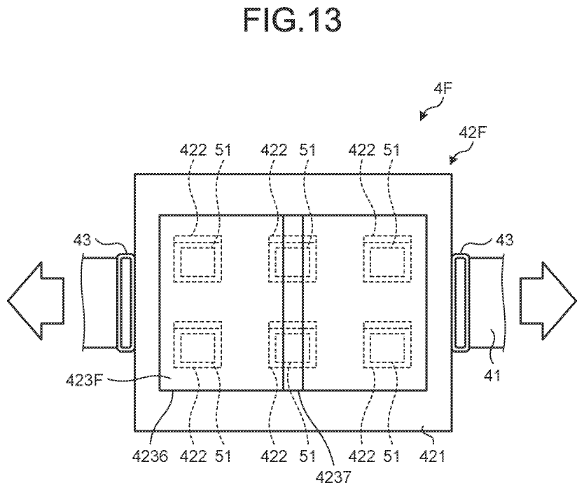

FIG. 13 is a diagram illustrating a configuration of an antenna holder according to a seventh embodiment of the disclosure. In the first embodiment described above, a case has been described as one example of a configuration in which the elastic force of the stretch portion 423 is fixed by one elastic force held by the material itself or is fixed by the arrangement location with respect to the main body 421; however, in the seventh embodiment, an adjustment portion that adjusts an elastic force of a stretch portion is disposed. An antenna holder 4F according to the seventh embodiment includes the belt portion 41, an antenna mounting portion 42F, and the connecting portions 43 that connect the belt portion 41 and the antenna mounting portion 42F. In the following, a configuration of a component (the antenna mounting portion 42F) that is different from that described above in the first embodiment will be described.

The antenna mounting portion 42F includes the above described main body 421, the above described antenna holding portions 422, and a stretch portion 423F that has a stretchable property and that is disposed on the main body 421. In the following, the stretch portion 423F that is different from the stretch portion described above in the first embodiment will be described.

The stretch portion 423F is formed by using a material having a stretchable property, such as a rubber or polyurethane elastic fiber. Similarly to the stretch portion 423, if the antenna holder 4 is attached to the subject 2, the stretch portion 423F is arranged on the side opposite to the subject 2 side relative to the main body 421. The stretch portion 423F includes a base portion 4236 that has a stretchable property and an adjustment portion 4237 that adjusts the length of the base portion 4236 in the first direction. The elastic force of the base portion 4236 is the same as the elastic force of, for example, the stretch portion 423 described above.

The adjustment portion 4237 is constituted by using, for example, a buckle. The base portion 4236 is inserted into the adjustment portion 4237 (buckle) and the length of the base portion 4236 in the first direction is adjusted.

In the seventh embodiment described above, in the antenna holder 4F that holds the reception antennas 51, the stretch portion 423F disposed on the main body 421 is elongated in accordance with the body shape of the subject 2 and allows the main body 421 to be brought into close contact with the subject 2. According to the seventh embodiment, it is possible to attach the reception antennas to the subject without reducing contact properties with respect to the subject. Furthermore, in the seventh embodiment, it is possible to adjust the degree of contact properties with respect to the subject 2 by adjusting the length of a base portion 4326 by the adjustment portion 4237.

### Eighth Embodiment

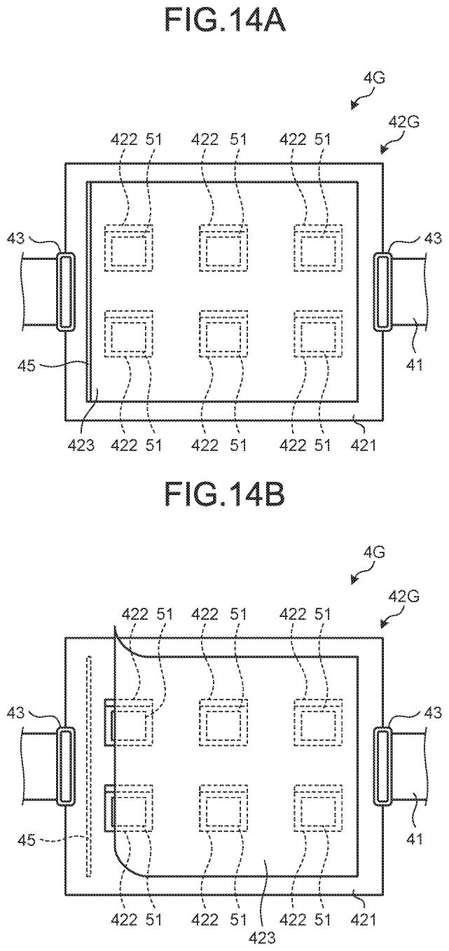

FIG. 14A and FIG. 14B are diagrams each illustrating a configuration of an antenna holder according to an eighth embodiment of the disclosure. In the first embodiment described above, a case has been described as one example of a configuration in which the stretch portion 423 is fixed to the main body 421; however, in the eighth embodiment, a part of the stretch portion 423 is able to be attached to and removed from the main body 421. An antenna holder 4G according to the eighth embodiment includes the belt portion 41, the antenna mounting portion 42G, the connecting portions 43, and a removable portion 45 that attaches and removes a part of the stretch portion 423 to and from the main body 421. In the following, a configuration of a component (the removable portion 45) that is different from that described above in the first embodiment will be described.

The removable portion 45 is formed by using, for example, a snap fastener, a zip fastener, or a hook-and-loop fasteners and attaches and removes one end of the stretch portion 423 in the first direction to and from the main body 421 (see FIG. 14B). In contrast, the other end of the stretch portion 423 in the first direction is fixed to the main body 421.

In the eighth embodiment described above, in the state in which the stretch portion 423 is attached to the main body 421 by the removable portion 45, similarly to the first embodiment, the stretch portion 423 disposed on the main body 421 is elongated in accordance with the body shape of the subject 2 and allows the main body 421 to be brought into close contact with the subject 2. According to the eighth embodiment, it is possible to attach the reception antennas to the subject without reducing contact properties with respect to the subject.

Furthermore, in the eighth embodiment, because one end of the stretch portion 423 is separated from the main body 421 by the removable portion 45, for example, if the antenna holding portions 422 are disposed on the side opposite to the subject 2 side relative to the main body 421, it is possible to expose the antenna holding portions 422 by turning over the stretch portion 423. By exposing the antenna holding portions 422 covered by the stretch portion 423, it is possible to easily insert and remove the reception antennas 51 to and from the antenna holding portions 422.

### Modification of Eighth Embodiment

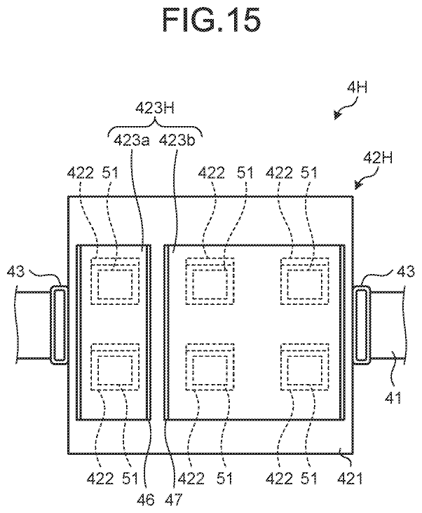

FIG. 15 is a diagram illustrating a configuration of an antenna holder according to a modification of the eighth embodiment of the disclosure. In the eighth embodiment described above, a case has been described as one example in which the single removable portion 45 is provided such that one end of the stretch portion 423 is able to be attached to and removed from the main body 421; however, in the modification, two removable portions 46 and 47 are provided. An antenna holder 4H according to the modification includes the belt portion 41, an antenna mounting portion 42H, the connecting portions 43, and the removable portions 46 and 47 that attach and remove a part of a stretch portion 423H to and from the main body 421. In the following, a configuration of components (the antenna mounting portion 42H and the removable portions 46 and 47) that are different from that described above in the first embodiment will be described.

The antenna mounting portion 42H includes the above described main body 421, the above described antenna holding portions 422, and the stretch portion 423H that has a stretchable property and that is disposed on the main body 421. The stretch portion 423H is formed by using a material having a stretchable property, such as a rubber or polyurethane elastic fiber. Similarly to the stretch portion 423, if the antenna holder 4 is attached to the subject 2, the stretch portion 423H is arranged on the side opposite to the subject 2 side relative to the main body 421. The stretch portion 423H includes a first stretch portion 423a that is disposed on one end of the first direction and a second stretch portion 423b that is disposed on the other end of the first direction.

Each of the removable portions 46 and 47 is formed by using, for example, a snap fastener, a zip fastener, or a hook-and-loop fastener.

The removable portion 46 is disposed on one end portion in the first direction and removes and attaches one end portion of the first stretch portion 423a from and to the main body 421. Furthermore, one end of the first stretch portion 423a in the first direction is fixed to the main body 421.

The removable portion 47 is disposed one end portion in the first direction and removes and attaches one end portion of the second stretch portion 423b from and to the main body 421. Furthermore, the other end of the second stretch portion 423b in the first direction is fixed to the main body 421.

The removable portions 46 and 47 are disposed at a location in which the first stretch portion 423a and the second stretch portion 423b act as double doors. Furthermore, the arrangement locations of the removable portions 46 and 47 are not limited to these as long as the first stretch portion 423a and the second stretch portion 423b act as double doors.

In the modification described above, in the state in which the first stretch portion 423a and the second stretch portion 423b are attached to the main body 421 by the removable portions 46 and 47, similarly to the eighth embodiment, the first stretch portion 423a and the second stretch portion 423b disposed on the main body 421 are elongated in accordance with the body shape of the subject 2 and allow the main body 421 to be brought into close contact with the subject 2. According to the modification, it is possible to attach the reception antennas to the subject without reducing contact properties with respect to the subject.

Furthermore, in the modification, because one end portion of the first stretch portion 423a and one end portion of the second stretch portion 423b are separated from the main body 421 by the removable portions 46 and 47, respectively, for example, if the antenna holding portions 422 are disposed on the side opposite to the subject 2 of relative to the main body 421, it is possible to expose the antenna holding portions 422 by turning over the stretch portion 423. By exposing the antenna holding portions 422 covered by the stretch portion 423, it is possible to easily insert and remove the reception antennas 51 to and from the antenna holding portions 422.

### Ninth Embodiment

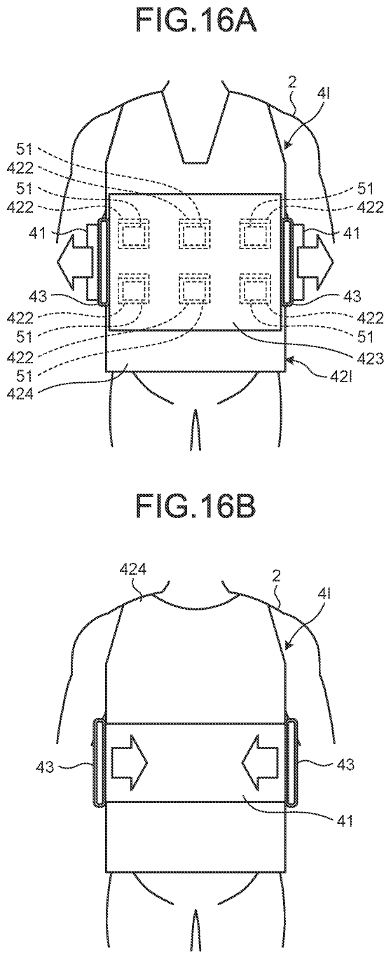

FIGS. 16A and 16B are diagrams each illustrating a configuration of an antenna holder according to a ninth embodiment of the disclosure. FIG. 16A is a diagram illustrating the subject 2 viewed from the front. FIG. 16B is a diagram illustrating the subject 2 viewed from the back. In the first embodiment described above, a case has been described as one example of a configuration in which the main body 421 has a sheet shape; however, in the ninth embodiment, a main body 424 has a shape so as to be capable for the subject 2 to wear. An antenna holder 4I according to the ninth embodiment includes the belt portion 41, an antenna mounting portion 42I, and the connecting portions 43. In the following, a configuration of component (the antenna mounting portion 42I) that is different from that described above in the first embodiment will be described.

The antenna mounting portion 42I includes the main body 424 worn by the subject 2, the above described antenna holding portions 422, and the above described stretch portion 423 that has a stretchable property and that is disposed on the main body 424. In the following, the main body 424 that is different from the main body described in the first embodiment will be described.

The main body 424 is a vest that is worn by the subject 2. The antenna holding portion 422 and the stretch portion 423 are disposed on the main body 424 on the front side. Furthermore, the connecting portions 43 are disposed on the main body 424, and the belt portion 41 is connected to the connecting portions 43. In the ninth embodiment, the connecting portions 43 are attached to the main body 424 on both sides of the stretch portion 423 in the first direction (the horizontal direction in FIG. 16A). Namely, the belt portions 41 are connected to the main body 424 across the stretch portion 423 in the first direction.

In the ninth embodiment described above, similarly to the first embodiment, the stretch portion 423 disposed on the main body 424 is elongated in accordance with the body shape of the subject 2 and allows an arrangement area of the antenna holding portions 422 disposed on the main body 424 to be brought into close contact with the subject 2. According to the ninth embodiment, it is possible to attach the reception antennas to the subject without reducing contact properties with respect to the subject.

### Modification of Ninth Embodiment

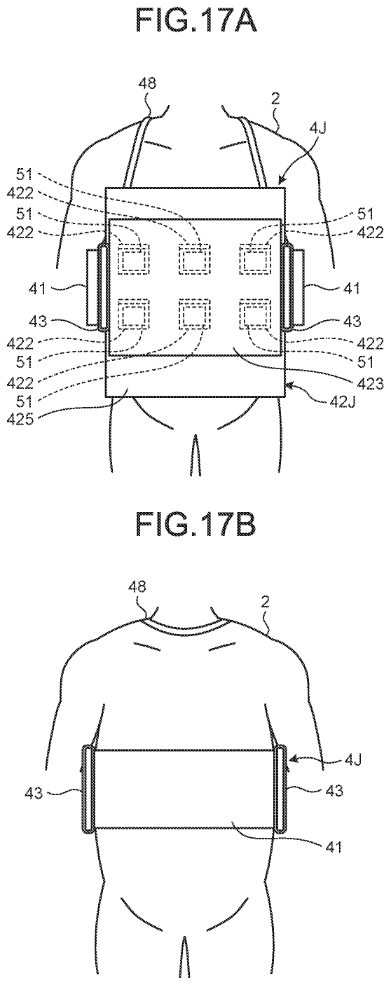

FIGS. 17A and 17B are diagrams each illustrating a configuration of an antenna holder according to a modification of the ninth embodiment of the disclosure. FIG. 17A is a diagram illustrating the subject 2 viewed from the front. FIG. 17B is a diagram illustrating the subject 2 viewed from the back. In the ninth embodiment described above, a case has been described as one example in which the main body 424 is a vest; however, in the modification, a main body 425 has a shape of an apron. An antenna holder 4J according to the modification includes the belt portion 41, an antenna mounting portion 42J, and the connecting portions 43. In the following, a configuration of component (the antenna mounting portion 42J) that is different from that described above in the first embodiment will be described.

The antenna mounting portion 42J includes the main body 425 worn by the subject 2, the above described antenna holding portion 422, and the above described stretch portion 423 that has a stretchable property and that is disposed on the main body 425. In the following, the main body 425 that is different from the main body described above in the ninth embodiment will be described.

The main body 425 is an apron worn by the subject 2. A string 48 through which the neck of the subject 2 is passed is attached to the main body 425.

The antenna holding portion 422 and the stretch portion 423 are disposed on the main body 425 on the front side. Furthermore, the connecting portions 43 are disposed on the main body 425 and to which the belt portion 41 is connected. In the modification, the connecting portions 43 are disposed at both ends of the main body 425 in the first direction (the horizontal direction in FIG. 17A). Namely, the belt portion 41 is connected to the main body 425 across the stretch portion 423 in the first direction.

In the modification described above, similarly to the ninth embodiment, the stretch portion 423 disposed on the main body 425 is elongated in accordance with the body shape of the subject 2 and allows the arrangement area of the antenna holding portions 422 disposed on the main body 425 to be brought into close contact with the subject 2. According to the modification, it is possible to attach the reception antennas to the subject without reducing contact properties to the subject.

In the above, detailed description of the preferred embodiments have been described; however, the disclosure is not limited by only the embodiments and the modifications described above. The disclosure is not limited to embodiments and the modifications as described above and may include various embodiment within the scope not departing from the technical concept as defined by the appended claims. In addition, configurations of the embodiments and modifications may also be combined appropriately.

Furthermore, in the first to the ninth embodiments described above, the capsule endoscope 3 has been described as an example of a device; however, the embodiments are not limited to this example. For example, it may also be possible to use a device that is introduced into the subject, that acquires pH information, and that outputs a radio signal.

Furthermore, in the first to the ninth embodiments described above, an example in which the stretch portion covers all of the arrangement area of the antenna holding portion has been described; however, it may also be possible to use a configuration in which a part of the antenna holding portion is exposed from the stretch portion as long as it is possible to bring the arrangement area of the antenna holding portion of the main body into close contact with the subject.

As described above, the antenna holders according to the disclosure is useful for attaching the reception antennas to the subject without reducing contact properties to the subject.

According to the disclosure, an advantage is provided in that it is possible to attach reception antennas to a subject without reducing contact properties to the subject.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An antenna holder comprising:
a plurality of antenna holding portions configured to hold reception antennas configured to receive a radio signal transmitted from a capsule endoscope;
a main body to which the antenna holding portions are fixed;
a stretch portion that is mounted on the main body, the stretch portion being configured to cover all of the antenna holding portions; and
a belt portion that is attached to the main body across the stretch portion in a first direction.

2. The antenna holder according to claim 1, wherein the stretch portion is configured to stretch in accordance with a tensile force that is applied to the main body and that is generated by the belt portion being attached to a subject.

3. The antenna holder according to claim 1, wherein a natural length of the stretch portion in the first direction is shorter than a length of a mounting area of the main body in the first direction, the mounting area being an area in which the stretch portion is mounted on the main body.

4. The antenna holder according to claim 1, wherein
the stretch portion includes
a first stretch portion that has a first elastic force,
a second stretch portion that has a second elastic force that is greater than the first elastic force, the second stretch portion being disposed on a side of a first end of the first stretch portion in a second direction that is orthogonal to the first direction, and
a third stretch portion that has a third elastic force that is greater than the first elastic force, the third stretch portion being disposed on a side of a second end of the first stretch portion in the second direction.

5. The antenna holder according to claim 1, wherein
the stretch portion includes
a base portion that has a stretchable property, and
a non-stretch portion that has a non-stretchable property and that is disposed at a central part of the base portion in the first direction.

6. The antenna holder according to claim 1, further comprising a reinforcement portion that is disposed between the main body and the belt portion, that has a non-stretchable property, and that has a length in a second direction that is orthogonal to the first direction, the length in the second direction being greater than a minimum length of an arrangement area in the second direction, the arrangement area being an area in which all of the antenna holding portions are arranged on the main body.

7. A capsule endoscope system comprising:
a capsule endoscope configured to be introduced into a subject;
an antenna holder configured to be attached to the subject, the antenna holder being configured to hold a plurality of reception antennas;
an antenna device that is mounted on the antenna holder and that includes the plurality of reception antennas configured to receive a radio signal transmitted from the capsule endoscope;
a reception device that is detachably connected to the antenna device, the reception device being configured to record or display data of the radio signal by performing a predetermined process on the radio signal received by the antenna device; and
an image processing apparatus configured to perform a process on or display image data of an image inside the subject captured by the capsule endoscope, wherein
the antenna holder includes
a plurality of antenna holding portions configured to hold the reception antennas,
a main body to which the antenna holding portions are fixed,
a stretch portion that is mounted on the main body, the stretch portion being configured to cover all of the antenna holding portions,
a belt portion that is attached to the main body across the stretch portion in a first direction.
